(19) United States
(12) Patent Application Publication
(10) Pub. No.: US 2020/0397948 A1
(43) Pub. Date: Dec. 24, 2020

(54) DENTAL ADHESIVE HYDROGELS AND USES THEREOF

(71) Applicant: The Regents of the University of California, Oakland, CA (US)

(72) Inventors: Alireza MOSHAVERINIA, Los Angeles, CA (US); Mohammad Mahdi HASANI-SADRABADI, Los Angeles, CA (US); Tara L. AGHALOO, Sherman Oaks, CA (US); Paul S. WEISS, Los Angeles, CA (US)

(73) Assignee: The Regents of the University of California, Oakland, CA (US)

(21) Appl. No.: 16/979,215

(22) PCT Filed: Mar. 11, 2019
(86) PCT No.: PCT/US19/21660

§ 371 Date: Sep. 9, 2020

Related U.S. Application Data
(60) Provisional application No. 62/641,147, filed on Mar. 9, 2018.

(51) Int. Cl. A61L 27/38; A61L 27/52; A61L 27/26

(52) U.S. Cl. CPC A61L 27/3865; A61L 27/3834; A61L 27/26; A61L 27/52

(57) ABSTRACT

A dental hydrogel composition comprising: (a) polysaccharide; (b) polydopamine conjugated to the polysaccharide, wherein between 5 and 35 percent of polysaccharide sugar moieties are conjugated to polydopamine; (c) RGD peptide coupled to the polysaccharide-poly-dopamine conjugate; and (d) moieties that are crosslinkable upon exposure to light coupled to the polysaccharide; wherein components (a)-(d) are disposed in the composition such that the hydrogel composition: exhibits an adhesive strength of at least 10 kPa upon cross linking of crosslinkable moieties; and exhibits an elasticity between 5 kPa and 100 kPa upon cross linking of crosslinkable moieties.

### STATEMENT AS TO RIGHTS TO INVENTIONS MADE UNDER FEDERALLY SPONSORED RESEARCH AND DEVELOPMENT

This invention was made with Government support under DE023825, awarded by the National Institutes of Health. The Government has certain rights in the invention.

### TECHNICAL FIELD

The present disclosure relates to dental adhesive hydrogel compositions and methods for making and using them.

### BACKGROUND OF THE INVENTION

Periodontitis is a prevalent, chronic, destructive inflammatory disease affecting tooth-supporting tissues in humans. Approximately 50% of Americans have some form of periodontal disease. Currently, no ideal treatment is available for periodontitis. The use of mesenchymal stem cells (MSCs) presents an advantageous therapeutic option for periodontal tissue engineering. Gingival mesenchymal stem cells (GMSCs) are of special interest as they are easily accessible in the oral cavity and readily found in discarded dental tissue samples. Biomaterials are widely used as cell delivery vehicles to direct stem cell differentiation toward desired phenotypes. In vitro, cultures of micron-scale cell aggregates recreate the biochemical and biophysical microenvironment of native tissues defined by cell-cell communications. Adhesion and retention of the biomaterial at the application site as well as its regenerative properties are vital factors for successful periodontal tissue regeneration. However, the major drawbacks of the current cell-laden biomaterials for periodontal tissue engineering are weak adhesion to the tissue, poor mechanical strength, fast/uncontrolled degradation, and absence of regenerative properties. Collagen has been used for periodontal tissue repair; however, poor mechanical properties, fast degradation, and difficulty of keeping the material at the site are its main drawbacks. Adhesive biomaterials (e.g., fibrin glue) are not promising cell delivery vehicles for periodontal tissue repair due to their lack of regenerative properties. Currently, there is no single biomaterial that combines the above-mentioned desired properties.

Accordingly, there is a need for improved materials and methods that can used to facilitate tissue engineering, for example periodontal tissue regeneration.

### SUMMARY OF THE INVENTION

In one aspect, the invention provides a dental hydrogel composition comprising:

(a) polysaccharide;

(b) polydopamine conjugated to the polysaccharide, wherein between 5 and 35% of polysaccharide sugar moieties are conjugated to polydopamine;

(c) RGD peptide coupled to the polysaccharide-polydopamine conjugate; and

(d) moieties that are crosslinkable upon exposure to light coupled to the polysaccharide;

wherein components (a)-(d) are disposed in the composition such that the hydrogel composition:

exhibits an adhesive strength of at least 10 kPa upon cross linking of crosslinkable moieties; and

exhibits an elasticity between 5 kPa and 100 kPa upon cross linking of crosslinkable moieties.

In another aspect the invention provides a bilayer dental hydrogel composition comprising:

a first layer and a second layer, each of the first layer and the second layer comprising the dental hydrogel composition provided herein;
(i) the first layer further comprising at least one growth fact selected from TGF-B1, TGF-B2, TGF-B3 or FGF; and
(ii) the second layer further comprising gingival mesenchymal stem cell aggregates having encapsulating surface functionalized hydroxyapatite molecules.

In a further aspect, the invention provides a method of encapsulating a gingival mesenchymal stem cells in a dental hydrogel composition comprising:
(i) disposing the gingival mesenchymal stem cells in the dental hydrogel composition provided herein; and
(ii) exposing (i) to light so that the moieties are crosslinked.

In another aspect, the invention provides a method of delivering gingival mesenchymal stem cells to periodontal tissue, the method comprising forming a bi-layered composition by:
forming a first layer of the bi-layered composition by:
(a) disposing TGF-B3 in the dental hydrogel composition provided herein;
(b) contacting the composition of (a) with periodontal tissue;
(c) crosslinking the moieties that are crosslinkable upon exposure to light by exposing the composition of (b) to light.

In an embodiment, the method further comprises:
forming a second layer of the bi-layered composition by:
(d) obtaining gingival mesenchymal stem cells;
(e) disposing the gingival mesenchymal stem cells in a hydrogel composition comprising, the hydrogel composition:
   (i) polysaccharide;
   (ii) polydopamine conjugated to the polysaccharide, wherein between 5 and 35% of polysaccharide sugar moieties are conjugated to polydopamine;
   (iii) RGD peptide coupled to the polysaccharide-polydopamine conjugate; and
   (iv) moieties that are crosslinkable upon exposure to light coupled to the polysaccharide;
   wherein components (i)-(iv) are disposed in the hydrogel composition such that the hydrogel composition:
   exhibits an adhesive strength of at least 10 kPa upon cross linking of crosslinkable moieties; and
   exhibits an elasticity between 5 kPa and 100 kPa upon cross linking of crosslinkable moieties;
(f) contacting the hydrogel composition of (e) with the first layer; and
(g) crosslinking the moieties that are crosslinkable upon exposure to light by exposing the composition of (f) to light;
   thereby delivering the gingival mesenchymal stem cells to the periodontal tissue.

In a further aspect, the invention provides a method for regenerating periodontal ligament-like and osteogenic tissues in a subject in need thereof, the method comprising:

(a) contacting a first layer of the bilayer dental hydrogel composition provide herein with periodontal tissue of the subject,

(b) exposing the first layer of the bi-layered hydrogel composition to light to crosslink the crosslinkable moieties;

(c) contacting the light exposed first layer of (b) with the second layer of the bilayer dental hydrogel composition; and

(d) exposing the second layer of the bi-layered hydrogel composition to light to crosslink the crosslinkable moieties.

In another aspect, the invention provides a method for treating periodontal disease in a subject in need thereof, the method comprising:

delivering gingival mesenchymal stem cells to periodontal tissue, the method comprising forming a bi-layered composition by:

forming a first layer of the bi-layered composition by:
(a) disposing TGF-B3 in a dental hydrogel composition comprising:
   (i) polysaccharide;
   (ii) polydopamine conjugated to the polysaccharide, wherein between 5 and 35% of polysaccharide sugar moieties are conjugated to polydopamine;
   (iii) RGD peptide coupled to the polysaccharide-polydopamine conjugate; and
   (iv) moieties that are crosslinkable upon exposure to light coupled to the polysaccharide;

wherein components (i)-(iv) are disposed in the composition such that the hydrogel composition:

exhibits an adhesive strength of at least 10 kPa upon cross linking of crosslinkable moieties; and

exhibits an elasticity between 5 kPa and 100 kPa upon cross linking of crosslinkable moieties.
(b) contacting the dental hydrogel composition of (a) with periodontal tissue;
(c) crosslinking the moieties that are crosslinkable upon exposure to light by exposing the composition of (b) to light.
   In an embodiment, the method further comprises:

forming a second layer of the bi-layered composition by:
(d) obtaining gingival mesenchymal stem cells;
(e) disposing the gingival mesenchymal stem cells in a hydrogel composition comprising, the hydrogel composition:
   (i) polysaccharide;
   (ii) polydopamine conjugated to the polysaccharide, wherein between 5 and 35% of polysaccharide sugar moieties are conjugated to polydopamine;
   (iii) RGD peptide coupled to the polysaccharide-polydopamine conjugate; and
   (v) moieties that are crosslinkable upon exposure to light coupled to the polysaccharide;
   wherein components (i)-(iv) are disposed in the hydrogel composition such that the hydrogel composition:
   exhibits an adhesive strength of at least 10 kPa upon cross linking of crosslinkable moieties; and
   exhibits an elasticity between 5 kPa and 100 kPa upon cross linking of crosslinkable moieties;
(f) contacting the hydrogel composition of (e) with the first layer; and
(g) crosslinking the moieties that are crosslinkable upon exposure to light by exposing the composition of (f) to light.

In one aspect, the invention provides a method for regenerating periodontal tissue in a subject in need thereof, the method comprising:

(a) contacting a first layer of a bilayer dental hydrogel composition with periodontal tissue of the subject, the bilayer dental hydrogel composition comprising:
(i) a first layer and
(ii) a second layer, each of the first layer and the second layer comprising a dental hydrogel composition, the dental hydrogel composition comprising:
   (1) polysaccharide;
   (2) polydopamine conjugated to the polysaccharide, wherein between 5 and 35% of polysaccharide sugar moieties are conjugated to polydopamine;
   (3) RGD peptide coupled to the polysaccharide-polydopamine conjugate; and
   (4) moieties that are crosslinkable upon exposure to light coupled to the polysaccharide;
wherein components (1)-(4) are disposed in the composition such that the hydrogel composition:
exhibits an adhesive strength of at least 10 kPa upon cross linking of crosslinkable moieties; and
exhibits an elasticity between 5 kPa and 100 kPa upon cross linking of crosslinkable moieties;
   (iii) the first layer further comprising at least one growth fact selected from TGF-B1, TGF-B2, TGF-B3 or FGF; and
   (iv) the second layer further comprising gingival mesenchymal stem cell aggregates having encapsulating surface functionalized hydroxyapatite molecules,
(b) exposing the first layer of the bi-layered hydrogel composition to light to crosslink the crosslinkable moieties;
(c) contacting the light exposed first layer of (b) with the second layer of the bilayer dental hydrogel composition; and
(d) exposing the second layer of the bi-layered hydrogel composition to light to crosslink the crosslinkable moieties.

The present invention relates to biological adhesives which are biodegradable, photocurable, and nontoxic, and useful for periodontal tissue regeneration and personalized precision oral care. As discussed in detail below, in order to address the clinical need for adhesive biomaterials for dental tissue regeneration, we engineered a new biomimetic visible light crosslinkable adhesive biodegradable biomaterial having tunable physical properties and ability to direct the growth encapsulated stem cells and regulate their differentiation toward osteogenic or periodontal ligament-like tissues. One illustrative embodiment of this adhesive hydrogel comprises a visible light crosslinkable dopamine-modified alginate hydrogel (VLC DA-Alg). To form one working embodiment of this novel adhesive, we first conjugate dopamine to alginate (Alg-DA) and methacrylated alginate-dopamine (also named “Alg-DA-MA” or “Alg-MA-DA”, which are used interchangeably herein) is then prepared by reacting Alg-DA with 2-aminoethyl methacrylate hydrochloride (AEMA). The resulted structure is further modified with peptides such as (Gly)4-Arg-Gly-Asp-Gly-Ser (G4RGDGS) to form VLC DA-Alg. As discussed below, this adhesive hydrogel is shear thinning and visible light crosslinkable and has tunable physical properties.

Illustrating the flexibility of certain constituents of the compositions of the invention, we have developed various hydrogels based on other polysaccharides in addition to alginate (e.g., hyaluronic acid, heparin, chitosan, chondroitin sulfate, carrageenan and the like). Such hydrogel-based adhesives provide a combination of properties that make them useful for periodontal tissue regeneration including: (1) suitable mechanical characteristics to ensure the proliferation and infiltration of cells, and tissue formation; (2) strong adhesion to surrounding tissues; (3) biodegradability with degradation rate relative to tissue ingrowth; (4) space maintainability; and (5) high in vivo biocompatibility. In illustrative embodiments of the invention, the adhesive polysaccharide hydrogels, can be crosslinked in less than 20 seconds after exposing to visible (blue) or ultraviolet (UV) light and adhere to both hard and soft tissues, for example native periodontal tissues (alveolar bone, gingival tissue, and root surfaces, see, e.g., FIGS. 3D-3F.

The invention disclosed herein has a number of embodiments. One embodiment of the invention is dental hydrogel composition formed from a selected constellation of materials that has been discovered to provide hydrogel with characteristics that are highly desirable for use in periodontal procedures, such as an ability to regenerate periodontal ligament (PDL)-like and osteogenic tissues. In illustrative embodiments of the invention, this hydrogel comprises polysaccharide such as alginate coupled to polydopamine, wherein a specified number/range of the polysaccharide sugar moieties on the polysaccharide are coupled to polydopamine. In these hydrogel compositions, RGD peptides as well as moieties that are crosslinked upon exposure to light are further disposed in the polysaccharide-polydopamine conjugate. In these compositions, the components are in amounts and formed in the composition so that the hydrogel composition exhibits a specified adhesive strength following cross linking of cross linkable moieties; and further exhibits a specified elasticity following cross linking of cross linkable moieties.

The dental hydrogel composition formed from the selected constellation of materials disclosed herein can include further agents, for example cells, growth factors, and agents that facilitate crosslinking of the moieties that are crosslinked upon exposure to light etc. Certain embodiments of the invention involve cured or crosslinked compositions, i.e., where a plurality of the crosslinkable moieties are crosslinked. In some embodiments of the invention, the composition comprises a constellation of components selected so that the dental material exhibits a desired property, such as shear thinning or an in vivo degradation profile of not biodegrading for at least 3 weeks, but biodegrading in less than 6 months.

In typical embodiments of the invention, the hydrogel composition comprises one or more layers, for example one or more layers that comprises growth factors and/or gingival mesenchymal stem cells. In one illustrative embodiment, the composition comprises a first layer formed from a selected constellation of materials that is disclosed herein, and also includes at least one growth factor such as TGF-B3; and a second layer formed from a selected constellation of materials that is disclosed herein and also includes gingival mesenchymal stem cell aggregates. Typically, these mesenchymal stem cell aggregates are selected to have encapsulating surface functionalized HAP molecules.

A related embodiment of the invention is a method of encapsulating a gingival mesenchymal stem cells in a dental hydrogel composition having a selected constellation of materials that is disclosed herein. In this method, gingival mesenchymal stem cells are disposed in the composition, and the composition is then exposed to light so that the crosslinkable moieties are crosslinked, thereby encapsulating gingival mesenchymal stem cells in the composition. In typical embodiments of this method, the hydrogel composition is formed to comprise a plurality of layers, including layers having molecules that modulate the growth and or differentiation of the encapsulated gingival mesenchymal stem cells.

Another embodiment of the invention is a method of delivering gingival mesenchymal stem cells to periodontal tissue. This method can comprise forming a bi-layered hydrogel composition. Such methods can include forming a first layer of a bi-layered composition by disposing TGF-B3 in a dental hydrogel composition having the selected constellation of materials that is disclosed herein, and then contacting this composition with periodontal tissue. This methodological embodiment the invention then includes crosslinking the moieties in this first layer that are crosslinked upon exposure to light by exposing this composition to light. This method can then include forming a second layer of the bi-layered composition by disposing the gingival mesenchymal stem cells in a dental hydrogel composition having the selected constellation of materials that is disclosed herein to form a second layer and then contacting this second layer with the first layer; and then crosslinkable moieties in the second layer by exposing the second layer to light. This produces a crosslinked bilayer composition that is used to deliver the gingival mesenchymal stem cells to periodontal tissue. Typically, in these embodiments, delivering the gingival mesenchymal stem cells to periodontal tissue in this way results in the regeneration of periodontal tissue.

Other objects, features and advantages of the present invention will become apparent to those skilled in the art from the following detailed description. It is to be understood, however, that the detailed description and specific examples, while indicating some embodiments of the present invention, are given by way of illustration and not limitation. Many changes and modifications within the scope of the present invention may be made without departing from the spirit thereof, and the invention includes all such modifications.

### BRIEF DESCRIPTION OF THE DRAWINGS

FIGS. 1A-1N show various embodiments of the present invention. FIG. 1A is a schematic illustration of chemical modification of alginate to make Alg-MA-DA-RGD which can be photopolymerized either via visible light (e.g., Eosin Y) or UV (e.g., Irgacure 2959)-based photoinitiators. FIG. 1B is a visualization of light cured synthesized hydrogel and FIG. 1C shows its microstructure via scanning electron microscopy (SEM), scale bar is 100 sm. FIGS. 1D-1E respectively show UV-Vis and H-NMR spectra of synthesized Alg-MA-DA-RGD. FIG. 1F shows a full factorial investigation of methacrylation degree (0-22%), and degree of dopamine conjugation (0-4 mol %) on swelling degree. FIG. 1G shows a cumulative amount of sample protein (BSA-FITC) after 48 h. FIG. 1H graphically shows in vitro degradation based on mass loss of the hydrogels with and without presence of dopamine/methacrylate groups. Using oxidized alginate, the degradation rate can be further reduced (ALG-MA-DAFastDeg). FIG. 1I shows hydrogel adhesiveness to rat gingival in accordance with embodiments described herein. FIG. 1J shows hydrogel adhesiveness to rat calvarial bone and periosteum in accordance with embodiments described herein. FIG. 1K shows hydrogel adhesiveness to human tooth root surface in accordance with embodiments described herein. FIG. 1L shows sequential images of tensile experiment on enamel adhesion. FIG. 1M shows the stress-strain curve to identify adhesion strength to pig's skin. FIG. 1N shows sequential images of tensile experiment on pig gingival tissues as related to FIG. 1L. The presented data are expressed as mean t SD. The results were statistically analyzed using unpaired t-tests. For all the tests, the threshold was set to p<0.05 for “statistically significant”, p<0.01 for “statistically very significant”.

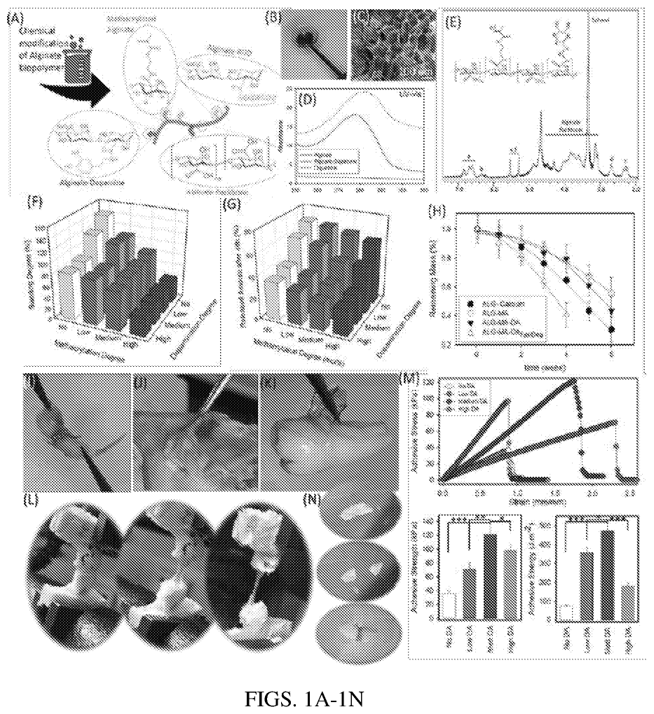

FIG. 2 shows embodiments of the herein provided adhesive hydrogel in dentistry applications for periodontal tissue regeneration.

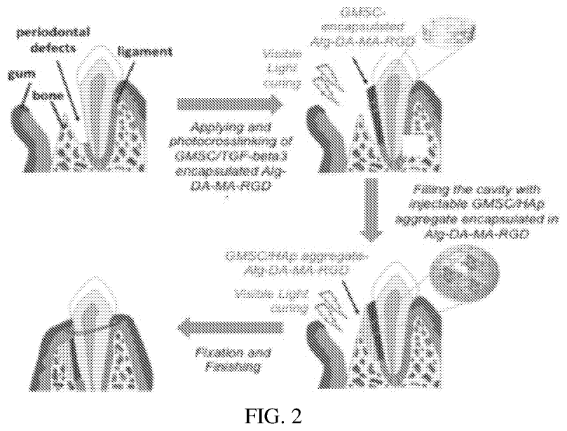

FIG. 3 shows schematic illustrations of the preparation of the herein provided adhesive hydrogel in accordance with embodiments described herein.

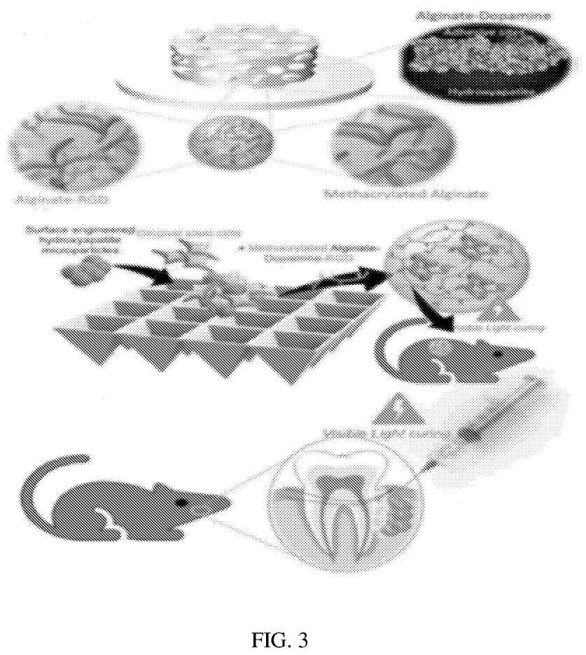

FIGS. 4A-4H show the biocompatibility of the herein provided adhesive hydrogel in accordance with embodiments described herein. FIGS. 4A-4B show in vitro biocompatibility of encapsulated gingiva-derived mesenchymal stem cells (“GMSCs”) inside hydrogel beads, in accordance with embodiments described herein. FIGS. 4C-4D respectively show live/dead staining fluorescence images of GMSC loaded Alginate RGD and Alg-MA-DA-RGD hydrogels, Scale bar: 500 μm. Quantitative live/dead results after one week of culturing in regular media indicated inside images. (p>0.05; Non-significant). FIGS. 4E-4F show in vivo biocompatibility of the herein provided hydrogel in accordance with embodiments described herein. FIG. 4E shows hematoxylin/eosin staining 7 days after subcutaneous implantation in wild type mice (asterisks indicate unresorbed alginate). FIG. 4F shows there were no signs of lymphocyte infiltration (CD3) after day 7 (scale bars: 200 μm). FIG. 4G shows whole blood analysis of mice after treating with various formulation of hydrogels in accordance with embodiments described herein. Values normalized to those for Alg-RGD. White blood cells: WBC (White blood cell), NE (Neutrophil), LY (Lymphocytes), MO (Monocytes), EO (Eosinophil), BA (Basophils). Red blood cell: HCT (Hematocrit), RBC (Red blood cell), HB (Hemoglobin), MCV (Mean corpuscular volume), MCH (Mean corpuscular hemoglobin), MCHC (Mean corpuscular hemoglobin concentration), RDW (Red cell Distribution Width), RSD (Reflex sympathetic dystrophy syndrome), RETIC (Reticulocyte). Platelets: PLT (Platelet count), MPV (Mean platelet volume), PDW (Platelet distribution width), and PCT (Plateletcrit). FIG. 4H shows comprehensive metabolic screening of mice after treating with various formulations of hydrogels in accordance with embodiments described herein. Values normalized to those for Alg-RGD. Liver function assessment: ALT (alanine aminotransferase), AST (aspartate aminotransferase), BUN (blood urea nitrogen), LDH (Lactate dehydrogenase). Kidney function assessment: CREAT (Creatinine), GLU (Glucose). Electrolytes: Calcium (CA), CO2 (carbon dioxide), MG (Magnesium), and PHOS (Phosphorus).

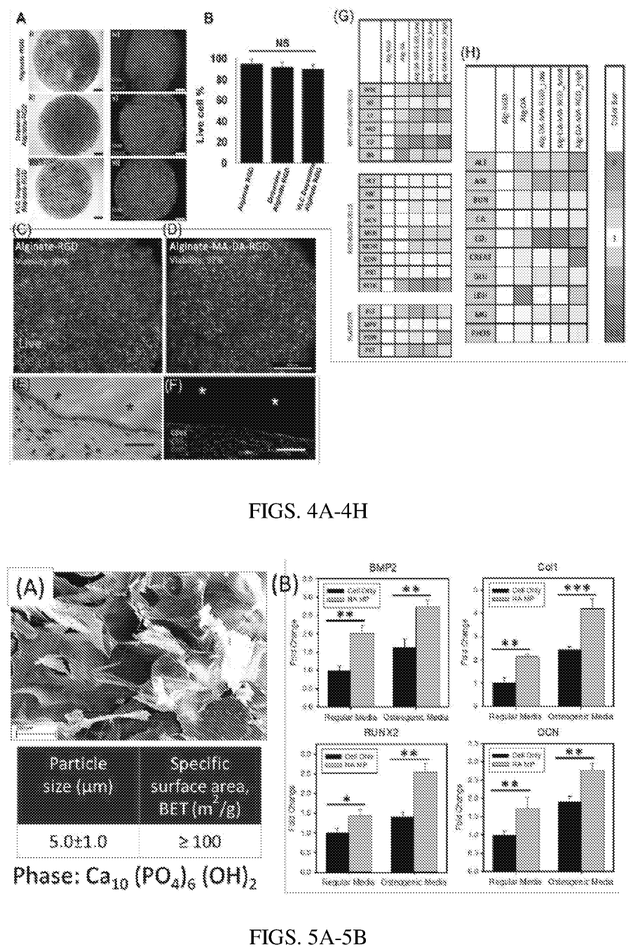

FIGS. 5A-5C show a Alg-MA-DA based hydrogel and expression of osteogenic genes from encapsulated GMSC cells within a Alg-MA-DA-RGD based hydrogel in accordance with embodiments described herein. FIGS. 5A-5B respectively show a scanning electron microscopy image of a Alg-MA-DA based hydrogel with 2 wt. % of microparticles in accordance with embodiments described herein and quantitative PCR demonstrating effects of Hap and bioactive glass microparticles on expression of osteogenic genes from encapsulated GMSC cells within a Alg-MA-DA-RGD based hydrogel in accordance with embodiments described herein. FIG. 5C shows Wnt antagonist sFRP-1 abolishes the effects of HA MPs on mesenchymal stem cells (“MSCs”).

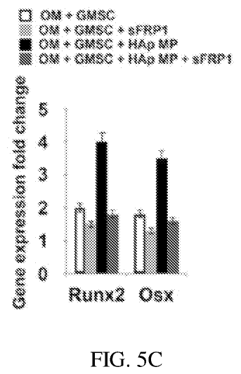

FIGS. 6A-6D show in vivo analyses of bone regeneration 8 weeks after subcutaneous implantation of 0.5 ml hydrogel in accordance with embodiments described herein into immunocompromised mice. FIG. 6A shows 3D reconstruction of micro-CT results in absence (upper panels), or presence of ca. 4×106 GMSCs (middle panels) or BMMSCs (lower panels) per mL of Alginate-RGD (±2 wt. % HA) or Alginate-DA-MA-RGD (±2 wt. % HA). FIG. 6B shows Faxitron digital in vivo two-dimensional X-rays of subcutaneously implanted hydrogels with GMSCs and BMMSCs. FIGS. 6C-6D respectively show quantified relative mineralized density as normalized to mouse bone density and bone volume (BV) fraction measurement derived from BV/total implanted volume (TV). The presented data are expressed as mean±SD. The results were statistically analyzed using unpaired t-tests. For all the tests, the threshold was set to p<0.05 for “statistically significant”, p<0.01 for “statistically very significant” and p<0.001 for “statistically extremely significant”. Statistical significance is indicated by * (significant), ** (very significant), and *** (extremely significant) for differences between samples with different formulations.

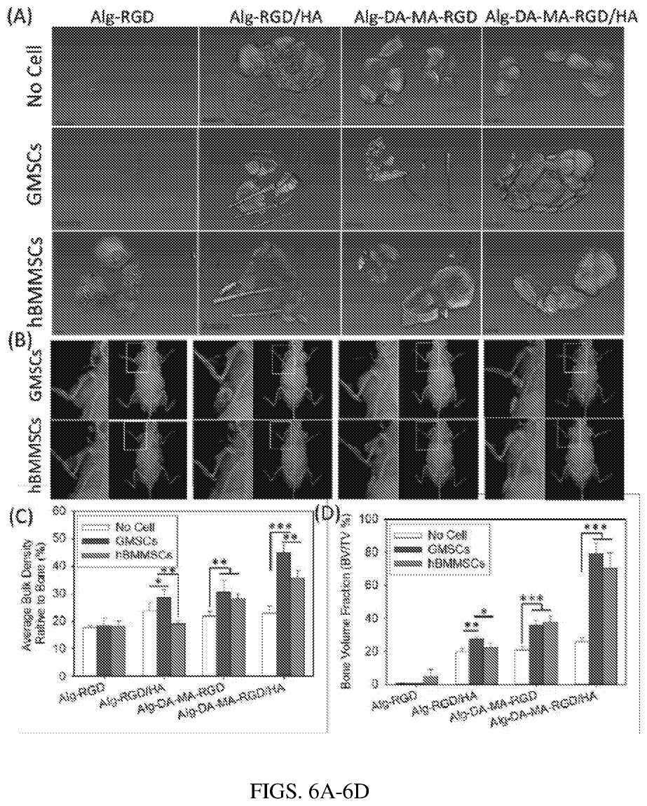

FIGS. 7A-7G show the effects of VCL DA-Alg adhesive hydrogel in accordance with embodiments described herein on ligament-like tissue regeneration and formation. FIG. 7A shows sustained release of TGF-β3 from the herein provided engineered VCL DA-Alg adhesive hydrogel. FIG. 7B shows in vitro differentiation of GMSCs encapsulated in VCL DA-Alg toward ligament-like tissues. Immunofluorescence staining against Tenomodulin (Tnmd) and Scleraxis (Scx) antibodies after four weeks of differentiation confirming the role of TGF-β3. FIG. 7C shows expression levels of Tnmd and Scx genes for encapsulated GMSCs after 4 weeks of differentiation evaluated by RTPCR. FIG. 7D shows Western blot analysis showing changes in the levels of expression of Tnmd during the differentiation of GMSCs toward ligament-like tissues. The level of Tnmd is elevated in the encapsulated GMSCs in the presence of TGF-β3. FIG. 7E shows ligament-like tissue formation in TGF-β3-loaded hydrogels in subcutaneous transplantation into nude mice confirmed through H&E, Masson's Trichrome staining, and polarized light microscopy. FIG. 7F shows positive immune-histochemical staining using antibodies against Tnmd and Scx. FIG. 7G shows semi-quantitative analysis of the percentage of MSCs positive for anti-Scx antibodies via immunohistochemical staining images in 5f. *P<0.05, **P<0.01.

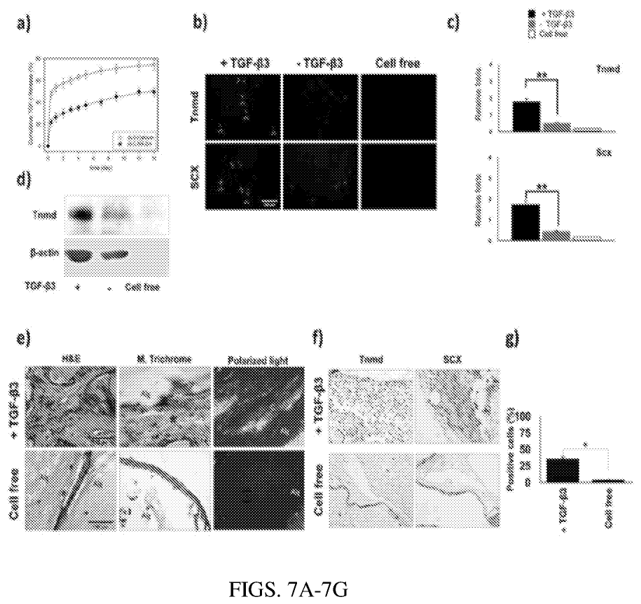

FIGS. 8A-8N show the effects on single GMSCs and aggregates of GMSCs encapsulated in Alg-DA-MA-RGD hydrogel without and with HAp microparticles FIGS. 8A-8B respectively show forced aggregation of GMSCs without and with HAp microparticles (cell:HA 1:1). FIGS. 8C-8D respectively show formation of cell GMSC without and with HAp GMSC spheroids inside microwells after 24 h of culture. FIGS. 8E-8F respectively show spheroids were removed from the wells and maintained in suspension culture. FIGS. 8G-8H respectively show light microscopy image of single GMSCs and aggregates of GMSCs encapsulated in Alg-DA-MA-RGD hydrogel; insets are live/dead staining of the encapsulated GMSCs are after 1 week of culturing in regular media. FIG. 8I shows quantitative live/dead assays showing the percentage of live cells on days 1 and 7. FIGS. 8J-8K respectively show Alizarin Red staining for single cell and cell aggregates of GMSCs encapsulated in Alg-DA-MA-RGD after 4 weeks of culturing in osteogenic media. insets are Xylenol orange staining for the mentioned conditions. FIG. 8L shows quantitative measurement of mineralization. FIG. 8M shows quantitative PCR demonstrating effects of HA microparticle presence at various rations to cells on expression of osteogenic genes. FIG. 8N shows HA microparticle loaded in hydrogels stimulates osteogenesis of GMSC aggregates via activation of Wnt/β-catenin signaling pathway. The presented data are expressed as mean t SD. The results were statistically analyzed using unpaired t-tests. For all the tests, the threshold was set to p<0.05 for “statistically significant”, p<0.01 for “statistically very significant” and p<0.001 for “statistically extremely significant”. Statistical significance is indicated by * (significant), ** (very significant), and *** (extremely significant) for differences between samples with different formulations. NS=not significant.

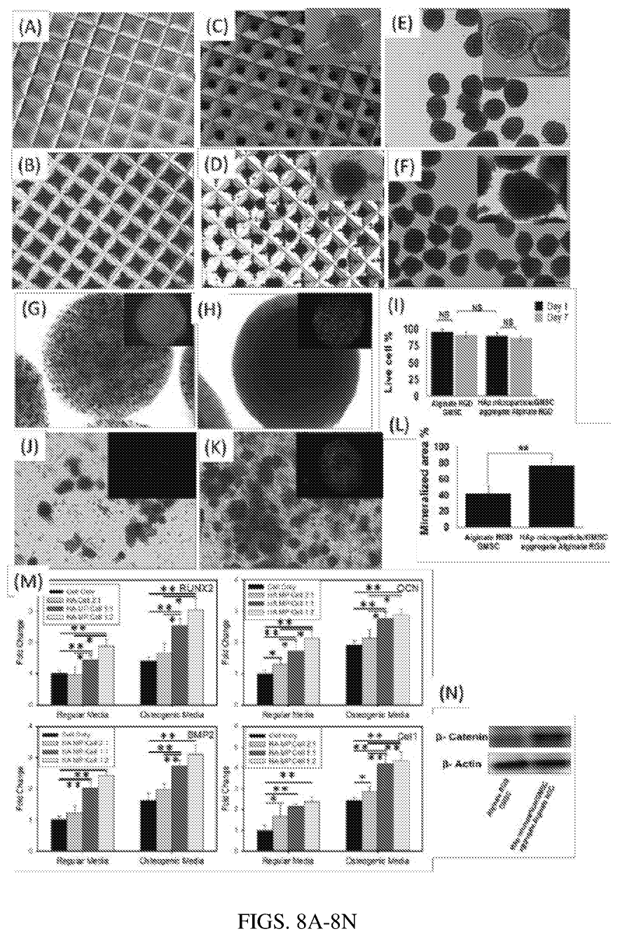

FIGS. 9A-9C show in vivo analyses of bone regeneration 8 weeks after subcutaneous injection of 0.25 ml hydrogel into immunocompromised mice. FIG. 9A shows 3D reconstruction and density mapping of micro-CT results in absence (upper panels), or presence of ca. 4×106 GMS single cells (middle panels) or ca. 4,000 GMS cell aggregates (lower panels) per mL of Alginate-RGD or Alginate-DA-MA-RGD. The hydrogels/aggregates contain equal amount of HA microparticles (HA MP:Cell 1:1) FIG. 9B shows quantified relative mineralized density as normalized to mouse bone density. FIG. 9C shows bone volume (BV) fraction measurement derived from BV/total implanted volume (TV).

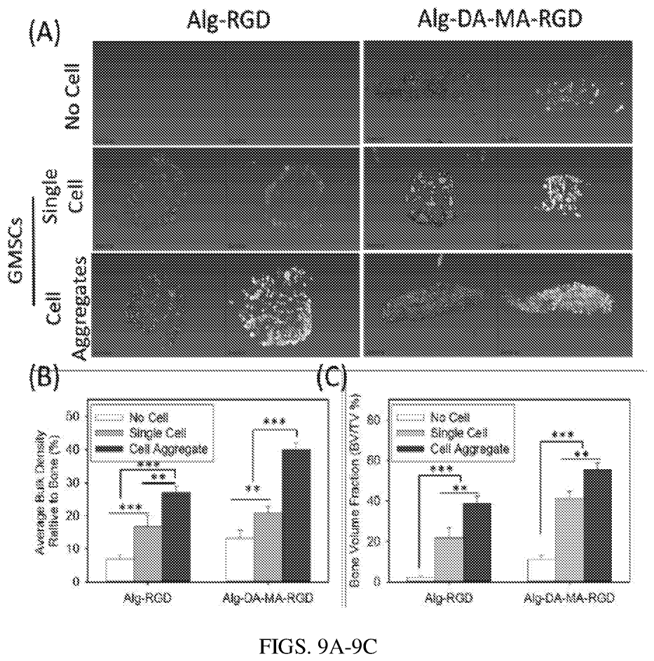

FIGS. 10A-10D show the effects of application of an embodiment of the herein provided adhesive hydrogel an animal model of P.g. induced peri-implantitis. FIG. 10A shows an animal model in rats. FIGS. 10B-10C show micro CT analyses before and after application of adhesive hydrogel showing complete bone fill at the defect site. FIG. 10D shows the inflammatory and anti-inflammatory profile of the defect side up to five weeks after application of the hydrogel biomaterials.

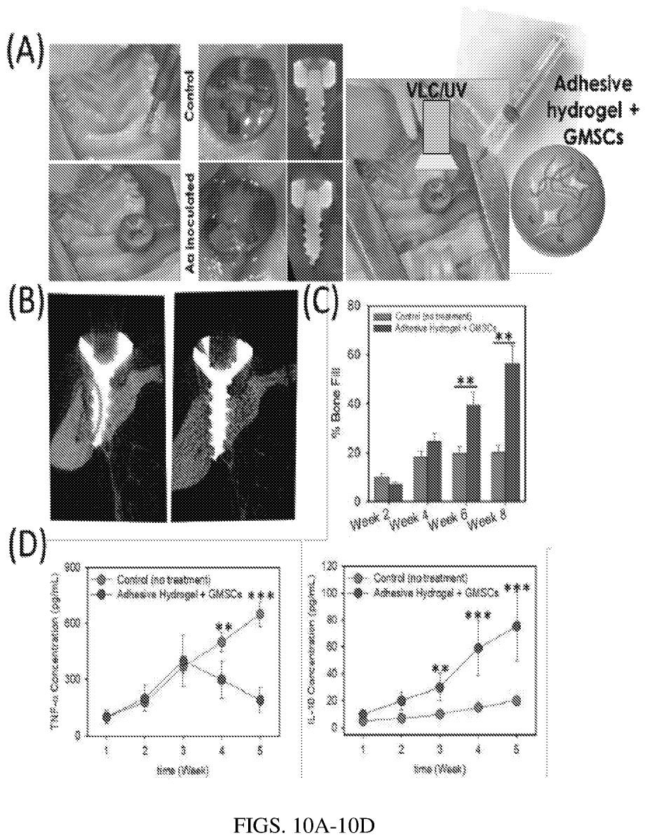

FIGS. 11A-11F show the effects of application of an adhesive hydrogel in accordance with embodiments described herein in an animal model. FIG. 11A shows a ligature induced periodontal disease model in rats. FIGS. 11B-11D show micro-CT reconstructed images of the rat maxilla: blue arrow points to normal alveolar bone and the yellow arrow points to periodontal bone loss after ligature. Green arrow shows bone regeneration after 8 weeks of application of Hap microparticle GMSC aggregate dopamine-modified alginate hydrogel in the bone loss site. Red and white arrows show the CEJ and bone crest levels, respectively. FIG. 11E shows micro-CT analysis of the rat maxilla showing the control site, the ligature site, and the defect size after application of the adhesive hydrogel. FIG. 11F shows a semi-quantitative analysis of the measurements (mm) from CEJ to the bone crest (unligated site, ligatured site, and 8 weeks after the application of adhesive hydrogel). The transverse plane illustrating the defect and CEJ to bone crest distance. *p<0.05, **p<0.01, ***p<0.001.

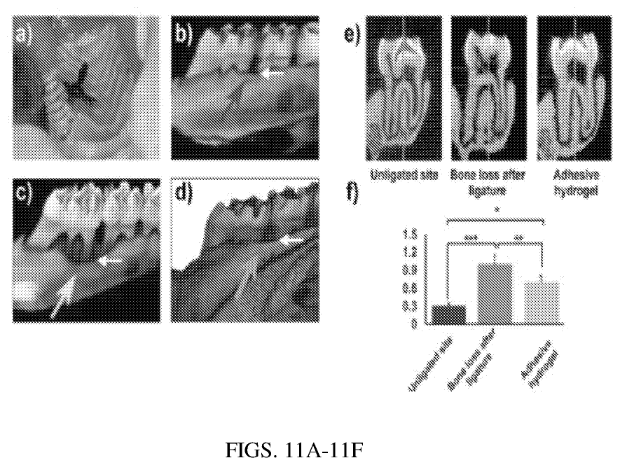

### DETAILED DESCRIPTION OF THE INVENTION

In the description of embodiments, reference may be made to the accompanying figures which form a part hereof, and in which is shown by way of illustration a specific embodiment in which the invention may be practiced. It is to be understood that other embodiments may be utilized and structural changes may be made without departing from the scope of the present invention. Many of the techniques and procedures described or referenced herein are well understood and commonly employed by those skilled in the art. Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art.

In one aspect, the invention provides a dental hydrogel composition comprising:

(a) polysaccharide;

(b) polydopamine conjugated to the polysaccharide, wherein between 5 and 35% of polysaccharide sugar moieties are conjugated to polydopamine;

(c) RGD peptide coupled to the polysaccharide-polydopamine conjugate; and

(d) moieties that are crosslinkable upon exposure to light coupled to the polysaccharide;

wherein components (a)-(d) are disposed in the composition such that the hydrogel composition:

exhibits an adhesive strength of at least 10 kPa upon cross linking of crosslinkable moieties; and

exhibits an elasticity between 5 kPa and 100 kPa upon cross linking of crosslinkable moieties.

In some embodiments, the dental hydrogel composition further comprises gingival mesenchymal stem cells.

In various embodiments, the polysaccharide is selected from the group consisting of alginate, hyaluronic acid, heparin, chitosan, chondroitin sulfate, and carrageenan.

In particular embodiments, the polydopamine coupled to the polysaccharide is methacrylated and the methacrylated polysaccharide has a degree of methacrylation that is between 1-22%.

In some embodiments, the dental hydrogel composition further comprises one or more agents that facilitate crosslinking of the moieties that are crosslinkable upon exposure to light.

In various embodiments of the dental hydrogel composition, the moieties are crosslinked.

In certain embodiments of the dental hydrogel composition, the composition exhibits at least one of:

shear thinning; and

an in vivo degradation profile of more than 4 weeks and less than 6 months.

In another aspect the invention provides a bilayer dental hydrogel composition comprising:

a first layer and a second layer, each of the first layer and the second layer comprising the composition provided herein;
(i) the first layer further comprising at least one growth fact selected from TGF-B1, TGF-B2, TGF-B3 or FGF; and
(ii) the second layer further comprising gingival mesenchymal stem cell aggregates having encapsulating surface functionalized hydroxyapatite molecules.

In some embodiments, the bilayer dental hydrogel composition further comprises gingival mesenchymal stem cells.

In certain embodiments of the bilayer dental hydrogel composition, the polysaccharide is selected from the group consisting of alginate, hyaluronic acid, heparin, chitosan, chondroitin sulfate, and carrageenan.

In various embodiments of the bilayer dental hydrogel composition, the polydopamine coupled to the polysaccharide is methacrylated and the methacrylated polysaccharide has a degree of methacrylation that is between 1-22%.

In some embodiments, the bilayer dental hydrogel composition further comprises one or more agents that facilitate crosslinking of the moieties that are crosslinkable upon exposure to light.

In various embodiments of the bilayer dental hydrogel composition, the moieties are crosslinked.

In certain embodiments of the bilayer dental hydrogel composition, the composition exhibits at least one of:

shear thinning;

an in vivo degradation profile of more than 4 weeks and less than 6 months;

a degree of dopamine conjugation that is between 5 wt. % and 35 wt. %.

In a further aspect, the invention provides a method of encapsulating a gingival mesenchymal stem cells in a dental hydrogel composition comprising:
(i) disposing the gingival mesenchymal stem cells in the dental hydrogel composition provided herein; and
(ii) exposing (i) to light so that the moieties are crosslinked.

In another aspect the invention provides a method of delivering gingival mesenchymal stem cells to periodontal tissue, the method comprising forming a bi-layered composition by: forming a first layer of the bi-layered composition by:
(a) disposing TGF-B3 in the dental hydrogel composition provided herein;
(b) contacting the composition of (a) with periodontal tissue;
(c) crosslinking the moieties that are crosslinkable upon exposure to light by exposing the composition of (b) to light.
   In some embodiments, the method further comprises:
forming a second layer of the bi-layered composition by:
(d) obtaining gingival mesenchymal stem cells;
(e) disposing the gingival mesenchymal stem cells in a hydrogel composition comprising, the hydrogel composition:
   (i) polysaccharide;
   (ii) polydopamine conjugated to the polysaccharide, wherein between 5 and 35% of polysaccharide sugar moieties are conjugated to polydopamine;
   (iii) RGD peptide coupled to the polysaccharide-polydopamine conjugate; and
   (iv) moieties that are crosslinkable upon exposure to light coupled to the polysaccharide;
   wherein components (i)-(iv) are disposed in the hydrogel composition such that the hydrogel composition:
   exhibits an adhesive strength of at least 10 kPa upon cross linking of crosslinkable moieties; and
   exhibits an elasticity between 5 kPa and 100 kPa upon cross linking of crosslinkable moieties;
(f) contacting the hydrogel composition of (e) with the first layer; and
(g) crosslinking the moieties that are crosslinkable upon exposure to light by exposing the composition of (f) to light;
   thereby delivering the gingival mesenchymal stem cells to the periodontal tissue.

In particular embodiments, delivering the gingival mesenchymal stem cells to periodontal tissue regenerates the periodontal tissue.

In some embodiments, the gingival mesenchymal stem cells exhibit more than 60% in vitro differentiation when disposed in the composition.

In another aspect, the invention provides a method for regenerating periodontal ligament-like and osteogenic tissues in a subject in need thereof, the method comprising:

(a) contacting a first layer of the bilayer dental hydrogel composition provide herein with periodontal tissue of the subject,

(b) exposing the first layer of the bi-layered hydrogel composition to light to crosslink the crosslinkable moieties;

(c) contacting the light exposed first layer of (b) with the second layer of the bilayer dental hydrogel composition; and

(d) exposing the second layer of the bi-layered hydrogel composition to light to crosslink the crosslinkable moieties.

In another aspect, the invention provides a method for treating periodontal disease in a subject in need thereof, the method comprising:

delivering gingival mesenchymal stem cells to periodontal tissue, the method comprising forming a bi-layered composition by:

forming a first layer of the bi-layered composition by:
(a) disposing TGF-B3 in a dental hydrogel composition comprising:
   (i) polysaccharide;
   (ii) polydopamine conjugated to the polysaccharide, wherein between 5 and 35% of polysaccharide sugar moieties are conjugated to polydopamine;
   (iii) RGD peptide coupled to the polysaccharide-polydopamine conjugate; and
   (iv) moieties that are crosslinkable upon exposure to light coupled to the polysaccharide;

wherein components (i)-(iv) are disposed in the composition such that the hydrogel composition:

exhibits an adhesive strength of at least 10 kPa upon cross linking of crosslinkable moieties; and

exhibits an elasticity between 5 kPa and 100 kPa upon cross linking of crosslinkable moieties.
(b) contacting the dental hydrogel composition of (a) with periodontal tissue;
(c) crosslinking the moieties that are crosslinkable upon exposure to light by exposing the composition of (b) to light.

In an embodiment, the method for treating periodontal disease in a subject in need thereof further comprises:

forming a second layer of the bi-layered composition by:
(d) obtaining gingival mesenchymal stem cells;
(e) disposing the gingival mesenchymal stem cells in a hydrogel composition comprising, the hydrogel composition:
   (i) polysaccharide;
   (ii) polydopamine conjugated to the polysaccharide, wherein between 5 and 35% of polysaccharide sugar moieties are conjugated to polydopamine;
   (iii) RGD peptide coupled to the polysaccharide-polydopamine conjugate; and
   (iv) moieties that are crosslinkable upon exposure to light coupled to the polysaccharide;
wherein components (i)-(iv) are disposed in the hydrogel composition such that the hydrogel composition:
exhibits an adhesive strength of at least 10 kPa upon cross linking of crosslinkable moieties; and
   exhibits an elasticity between 5 kPa and 100 kPa upon cross linking of crosslinkable moieties;
(f) contacting the hydrogel composition of (e) with the first layer; and
(g) crosslinking the moieties that are crosslinkable upon exposure to light by exposing the composition of (f) to light.

In particular embodiments, delivering the gingival mesenchymal stem cells to periodontal tissue regenerates the periodontal tissue.

In some embodiments, the gingival mesenchymal stem cells exhibit more than 60% in vitro differentiation when disposed in the composition.

In another aspect, the invention provides a method for regenerating periodontal tissue in a subject in need thereof, the method comprising:

(a) contacting a first layer of a bilayer dental hydrogel composition with periodontal tissue of the subject, the bilayer dental hydrogel composition comprising:
(i) a first layer and
(ii) a second layer, each of the first layer and the second layer comprising a dental hydrogel composition, the dental hydrogel composition comprising:
   (1) polysaccharide;
   (2) polydopamine conjugated to the polysaccharide, wherein between 5 and 35% of polysaccharide sugar moieties are conjugated to polydopamine;
   (3) RGD peptide coupled to the polysaccharide-polydopamine conjugate; and
   (4) moieties that are crosslinkable upon exposure to light coupled to the polysaccharide;
wherein components (1)-(4) are disposed in the composition such that the hydrogel composition:
exhibits an adhesive strength of at least 10 kPa upon cross linking of crosslinkable moieties; and
exhibits an elasticity between 5 kPa and 100 kPa upon cross linking of crosslinkable moieties;
   (iii) the first layer further comprising at least one growth fact selected from TGF-B1, TGF-B2, TGF-B3 or FGF; and
(iv) the second layer further comprising gingival mesenchymal stem cell aggregates having encapsulating surface functionalized hydroxyapatite molecules,

(b) exposing the first layer of the bi-layered hydrogel composition to light to crosslink the crosslinkable moieties;

(c) contacting the light exposed first layer of (b) with the second layer of the bilayer dental hydrogel composition; and

(d) exposing the second layer of the bi-layered hydrogel composition to light to crosslink the crosslinkable moieties.

In some embodiments of the method for regenerating periodontal tissue in a subject in need thereof, the polysaccharide is selected from the group consisting of alginate, hyaluronic acid, heparin, chitosan, chondroitin sulfate, and carrageenan.

In various embodiments of the method, the polydopamine coupled to the polysaccharide is methacrylated and the methacrylated polysaccharide has a degree of methacrylation that is between 1-22%.

In certain embodiments of the method, the dental hydrogel composition further comprises one or more agents that facilitate crosslinking of the moieties that are crosslinkable upon exposure to light.

In some embodiments of the method, the dental hydrogel composition comprises moieties that are crosslinked.

The invention disclosed herein has a number of embodiments. One embodiment of the invention is dental hydrogel composition formed from a selected constellation of materials that has been discovered to provide hydrogel with characteristics that are highly desirable for use in periodontal procedures, such as an ability to regenerate periodontal ligament (PDL)-like and osteogenic tissues. In illustrative embodiments of the invention, this hydrogel comprises polysaccharide such as alginate coupled to polydopamine, wherein between 5% and 25% (or 30% or 35%) of polysaccharide sugar moieties are coupled to polydopamine. In these hydrogel compositions, RGD peptides (see, e.g., Kung Biomed Mater Eng. 2018; 29(2):241-251, which is incorporated by reference in its entirety) as well as moieties that are crosslinked upon exposure to light are further disposed in the polysaccharide-polydopamine conjugate. In these compositions, the components are in amounts and formed in the composition so that the hydrogel composition exhibits an adhesive strength of at least 10 kPa and up to 100 kPa (typically >25 kPa) following cross linking of cross linkable moieties; and further exhibits an elasticity between 5 kPa and 100 kPa (e.g., between 10 kPa and 40 kPa) following cross linking of cross linkable moieties.

The dental hydrogel composition formed from the selected materials disclosed herein can include further agents, for example one or more agents that facilitate crosslinking of the moieties that are crosslinked upon exposure to light. Certain embodiments of the invention involve cured or crosslinked compositions, i.e., where a plurality of the crosslinkable moieties are crosslinked. In some embodiments of the invention, the composition comprises selected components provided herein so that the dental material exhibits at least one of: shear thinning; an in vivo degradation profile of not biodegrading for at least 4 weeks, but biodegrading in less than 6 months; a methacrylated polysaccharide (e.g., alginate) with a degree of methacrylation that is between 1-22%; and/or polysaccharide having a degree of dopamine conjugation that is between 1-4 mol % (or between 5 and 35 wt %).

In typical embodiments of the invention, the hydrogel composition comprises one or more layers, for example one or more layers that comprises growth factors and/or gingival mesenchymal stem cells. In one illustrative embodiment, the composition comprises a first layer formed from the selected materials that are disclosed herein, and also includes at least one growth factor such as TGF-B1, TGF-B2, TGF-B3 or FGF; and a second layer formed from a selected materials that are disclosed herein and also includes gingival mesenchymal stem cell aggregates (e.g., those obtained from a patient via a tissue punch). Typically, these mesenchymal stem cell aggregates are formed or selected to have encapsulating surface functionalized HAP molecules (see, e.g., Shi et al., Colloids Surf B Biointerfaces. 2017 Jul. 1; 155:477-486, which is incorporated by reference in its entirety).

A related embodiment of the invention is a method of encapsulating a gingival mesenchymal stem cells in a dental hydrogel composition having a selected materials that are disclosed herein. In this method, gingival mesenchymal stem cells are disposed in the composition, and the composition is then exposed to light so that the crosslinkable moieties are crosslinked, thereby encapsulating gingival mesenchymal stem cells in the composition. In typical embodiments of this method, the hydrogel composition is formed to comprise a plurality of layers, including layers having molecules that modulate the growth and or differentiation of the encapsulated gingival mesenchymal stem cells.

Another embodiment of the invention is a method of delivering gingival mesenchymal stem cells to periodontal tissue. This method can comprise forming a bi-layered hydrogel composition. Such methods can include forming a first layer of a bi-layered composition by disposing a growth factor such as TGF-B3 in a dental hydrogel composition having the selected materials that are disclosed herein, and then contacting this composition with periodontal tissue. This methodological embodiment the invention then includes crosslinking the moieties in this first layer that are crosslinked upon exposure to light by exposing this composition to light. This method can then include forming a second layer of the bi-layered composition by disposing the gingival mesenchymal stem cells in a dental hydrogel composition having the selected materials that are disclosed herein to form a second layer and then contacting this second layer with the first layer; and then crosslinkable moieties in the second layer by exposing the second layer to light. This produces a crosslinked bilayer composition that is used to deliver the gingival mesenchymal stem cells to periodontal tissue. Typically, in these embodiments, delivering the gingival mesenchymal stem cells to periodontal tissue results in the regeneration of periodontal tissue. Optionally, the gingival mesenchymal stem cells exhibit more than 40%, 50%, or 60% in vitro differentiation when disposed in compositions of the invention.

### ASPECTS AND ELEMENTS OF THE INVENTION

Dental restorative materials are known for restoring the function, morphology and integrity of dental structures damaged by physical damage or caries-related decay of enamel and/or dentin. Optimal dental restorative materials have high biocompatibility, good mechanical properties and mechanical and chemical resistance over a long period of time.

To address the limitations observed with conventional biomaterials for use in applications such as periodontal tissue engineering, we have engineered an adhesive hydrogel based on a visible light crosslinkable dopamine-modified alginate hydrogel with tunable physical properties and ability to regenerate periodontal ligament (PDL)-like and osteogenic tissues. Adhesive hydrogels have been developed through modification of polymers with dopamine. Cell aggregates/microparticles (polyelectrolyte-coated hydroxyapatite (HAp)) and growth factors (transforming growth factor β3 (TGF-β3)) have been used for differentiation of MSCs toward osteogenic and PDL-like tissues, respectively. However, combining these advancements for dental applications has not been explored. We have developed an adhesive hydrogel based on visible light crosslinkable dopamine-modified alginate-RGD (VLC DA-Alg), as a GMSC delivery vehicle for periodontal tissue regeneration.

In this novel treatment modality, GMSC-laden VLC DA-Alg hydrogels containing TGF-β3 are first delivered and photo crosslinked to form PDL-like tissue. A second layer of VLC DA-Alg hydrogel containing GMSC aggregates encapsulating surface functionalized HAp microparticles are then delivered and photopolymerized to repair alveolar bone tissue. Our engineered adhesive has strong adhesion to the periodontal tissues due to dopamine functionalization, maintain the space and prevent the formation of long junctional epithelium, facilitating PDL and bone tissue formation at the defect site. It also provides an appropriate microenvironment to regulate the fate of the encapsulated GMSCs toward periodontal tissues. We believe that direct cell-cell contact and the presence of osteoinductive microparticles can have a synergistic effect on in situ bone formation.

In this application we have developed an adhesive visible-light crosslinkable dopamine-modified alginate hydrogel with osteoconductive properties and the ability to regenerate PDL-like tissues. These compositions are useful in a variety of contexts, and the present invention further relates to the use of the compositions in dental and bone applications, in particular as a dental filling material, a dental coating material, a dental bonding cement, a bone cement and a bone replacing material.

### EXAMPLES

### Example 1

### Synthesis of Visible Light Crosslinkable (VLC) DA-Alg Hydrogel

To make VLC DA-Alg hydrogels, alginate (Protantol LF 10/60; FMC Biopolymer) were oxidized and purified, as reported before by Moshaverinia A, et al., Alginate hydrogel as a promising scaffold for dental-derived stem cells: an in vitro study. Journal of Materials Science: Materials in Medicine. 2012; 23(12):3041-51, which is incorporated herein by reference in its entirety. Alginate-dopamine was synthesized by activating the carboxy groups of alginate and reacting them with the amino groups on dopamine as described by Kastrup C J, et al., Painting blood vessels and atherosclerotic plaques with an adhesive drug depot. Proceedings of the National Academy of Sciences. 2012; 109(52):21444-9. doi: 10.1073/pnas.1217972110, which is incorporated herein by reference in its entirety. Alginate (0.75% w/v) was dissolved in 100 mM 2-(N-morpholino) ethanesulfonic acid (MES) buffer consisting at pH 6.1. N2 gas was bubbled through the solution during dissolution to remove oxygen gas. After overnight stirring at room temperature, N-(3-dimethylaminopropyl)-N-ethylcarbodiimide hydrochloride (EDC) and N-hydroxysuccinimide (NHS) was dissolved in MES buffer and added to the alginate solution. After mixing for 15 min, dopamine was dissolved in MES buffer and added to the alginate reaction mixture (molar ratio of NHS:EDC:dopamine 1.2:3:1). The reaction mixture will be stirred for 3 h, then dialyzed against PBS (MWCO 6-8 kDa) and concentrated using 10 kDa CentriPrep centrifugal filters. Then the mixture will be freeze-dried (we anticipate 50-70% yield). Incorporation of dopamine into alginate will be confirmed by UV-Visible, FTIR, and NMR spectroscopy measurements. Methacrylated alginate will be prepared by reacting alginate with 2-aminoethyl methacrylate (Table 1). Experimental design for adhesive physical properties optimization. hydrochloride (AEMA), as described by Bencherif S A, et al., Injectable cryogel-based whole-cell cancer vaccines. Nature communications. 2015; 6, which is incorporated herein by reference in its entirety. Alg-DA (0.75% w/v) in 100 mM MES buffer (pH 6.5) will be reacted with EDC and NHS (3:1 molar ratio) for 15 min. Next, AEMA (molar ratio of NHS:EDC:AEMA 1.2:3:1) will be added to the product and the solution will be stirred at room temperature for 24 h. The mixture will be dialyzed, purified, and characterized as above. VLC DA-Alg will be synthesized by coupling amine-terminated G4RGDGS peptide to carboxylic groups of alginate via EDC/NHS chemistry, as reported previously. We will then engineer adhesive hydrogels by mixing various concentrations of alginate and photoinitiator and exposing the mixture to visible light to crosslink the methacrylate functional groups presented on the VLC DA-Alg. We will use Eosin Y as an initiator, triethanolamine (TEA) as a co-initiator, and vinyl caprolactam (VC) as a catalyst to initiate reaction through exposing to blue-green light (450-550 nm, Xenon source) at 100 mW/cm2 for 10-60 sec, as described by Cleophas R T, et al., Characterization and activity of an immobilized antimicrobial peptide containing bactericidal PEG-hydrogel. Biomacromolecules. 2014; 15(9):3390-5. doi: 10.1021/bm500899r. PubMed PMID: 25109707, which is incorporated herein by reference in its entirety. We have previously used this system to generate visible light crosslinkable dopamine conjugated alginate hydrogel. We will study the effect of VLC Alg-DA concentration, degrees of conjugations with dopamine and methacrylation, Eosin-Y/TEAVC concentration, and light exposure time on the physical properties of the engineered VLC DAAlg hydrogels. In our preliminary studies, we have used hydrogels formed by conjugation of dopamine (3,4-dihydroxyphenethylamine) to alginate followed by methacrylation and then peptide (G4RGDGS) conjugation, followed by exposure to visible light in the presence of a photoinitiator (Eosin Y). This alginate-based hydrogel can be crosslinked via other methods, e.g., addition of Ca2+-reach media, or alternatively, dopamine residues oxidize easily by both chemical and enzymatic means. Our preliminary results showed that VLC Alg-DA gels had favorable mechanical properties and also strongly adhered to native tissues with adhesion strength higher than that of a commercially available adhesive. VLC DA-Alg hydrogel also supported cellular viability A three-factor, three-level Box-Behnken design has been used to explore responses with Design Expert (DE, Version 7.1, Stat-Ease Inc.). This cubic design was characterized by set of points lying at the midpoint of each edge of a multi-dimensional cube and center point replicates, whereas the “missing corners” help the experimenter to avoid the combined factor extremes (36-40). Five factors (independent variables) were considered: alginate concentration, dopamine conjugation, methacrylation degree, VC concentration, and light exposure time. A total of 46 experiments were carried out to optimize the physical properties of the engineered hydrogels. Based on these experiments, we have selected the candidates with suitable properties. As we plan to use the selected formulations for periodontal tissue regeneration, the engineered hydrogels should have suitable elasticity (10-40 kPa), adhesion to periodontal tissues (>25 kPa adhesion strength), degradation profile (more than 3 weeks and less than 6 months), and biocompatibility (above 90% cell viability). Therefore, our objective has been to select the formulations that meet these targets while presenting acceptable regenerative properties (more than 60% in vitro differentiation).

TABLE 1
Experimental design for adhesive physical properties optimization.

| | Input | | | |
|---|---|---|---|---|
| | Low | High | Low | High |
| | Actual | Actual | Coded | Coded |
| Independent Variables | Value | Value | Value | Value |
| Degree of methacrylation | 0 | 15 | −1 | +1 |
| (mol %) | | | | |
| Degree of dopamine | 0 | 4.0 | −1 | +1 |
| conjugation (mol %) | | | | |
| Photoinitiator | 0.5 | 2.0 | −1 | +1 |
| concentration (wt/v %) | | | | |
| Light exposure time (s) | 10 | 60 | −1 | +1 |
| Polymer Concentration | 0.5 | 5 | −1 | +1 |
| (wt/v %) | | | | |

The present invention provides dental adhesive compositions formed by the herein provided components that provide them with a number of highly desirable properties. Hydrogel-based adhesives such as fibrin and collagen are known in the art for sealing tissues or coating of implants to improve their adhesion to the surrounding tissues. However, poor mechanical properties and adhesion to the tissues in wet environments, are limitations for the successful implementation of these typical adhesives that are used in clinics. Inspired by the superior ability of mussels to adhere to wet surfaces, the critical role of the L-DOPA amino acid in adhesiveness has been identified and harnessed in this invention. The formation of a dopamine modified hydrogels with strong adhesion to periodontal tissues in the presence of blood or saliva is a paradigm shift in biomaterials science that enables the maintenance of the hydrogel in a defect site while new tissue forms. The compositions of the invention can be used for example as a dental cement, as a bone cement, as a dental repair material, and as a bone repair material.

All publications mentioned herein (e.g., those above and Lee et al., Science, 2007 Oct. 19; 318(5849): 426-430 and U.S. Patent Publication No. 20160331564) are incorporated herein by reference in their entireties to disclose and describe the methods and/or materials in connection with which the publications are cited. Publications cited herein are cited for their disclosure prior to the filing date of the present application. Nothing here is to be construed as an admission that the inventors are not entitled to antedate the publications by virtue of an earlier priority date or prior date of invention. Further, the actual publication dates may be different from those shown and require independent verification.

This concludes the description of the illustrative embodiments of the present invention. The foregoing description of one or more embodiments of the invention has been presented for the purposes of illustration and description. It is not intended to be exhaustive or to limit the invention to the precise form disclosed. Many modifications and variations are possible in light of the above teaching.

What is claimed is:

1. A dental hydrogel composition comprising:
(a) polysaccharide;
(b) polydopamine conjugated to the polysaccharide, wherein between 5 and 35% of polysaccharide sugar moieties are conjugated to polydopamine;
(c) RGD peptide coupled to the polysaccharide-polydopamine conjugate; and
(d) moieties that are crosslinkable upon exposure to light coupled to the polysaccharide;
wherein components (a)-(d) are disposed in the composition such that the hydrogel composition:
exhibits an adhesive strength of at least 10 kPa upon cross linking of crosslinkable moieties; and
exhibits an elasticity between 5 kPa and 100 kPa upon cross linking of crosslinkable moieties.

2. The dental hydrogel composition of claim 1, further comprising gingival mesenchymal stem cells.

3. The dental hydrogel composition of claim 1, wherein the polysaccharide is selected from the group consisting of alginate, hyaluronic acid, heparin, chitosan, chondroitin sulfate, and carrageenan.

4. The dental hydrogel composition of claim 1, wherein the polydopamine coupled to the polysaccharide is methacrylated and the methacrylated polysaccharide has a degree of methacrylation that is between 1-22%.

5. The dental hydrogel composition of claim 1, further comprising one or more agents that facilitate crosslinking of the moieties that are crosslinkable upon exposure to light.

6. The dental hydrogel composition of claim 1, wherein the moieties are crosslinked.

7. The dental hydrogel composition of claim 1, wherein the composition exhibits at least one of:
shear thinning; and
an in vivo degradation profile of more than 4 weeks and less than 6 months.

8. A bilayer dental hydrogel composition comprising:
a first layer and a second layer, each of the first layer and the second layer comprising the composition of claim 1;
(i) the first layer further comprising at least one growth fact selected from TGF-B1, TGF-B2, TGF-B3 or FGF; and
(ii) the second layer further comprising gingival mesenchymal stem cell aggregates having encapsulating surface functionalized hydroxyapatite molecules.

9. The bilayer dental hydrogel composition of claim 8, further comprising gingival mesenchymal stem cells.

10. The bilayer dental hydrogel composition of claim 8, wherein the polysaccharide is selected from the group consisting of alginate, hyaluronic acid, heparin, chitosan, chondroitin sulfate, and carrageenan.

11. The bilayer dental hydrogel composition of claim 8, wherein the polydopamine coupled to the polysaccharide is methacrylated and the methacrylated polysaccharide has a degree of methacrylation that is between 1-22%.

12. The bilayer dental hydrogel composition of claim 8, further comprising one or more agents that facilitate crosslinking of the moieties that are crosslinkable upon exposure to light.

13. The bilayer dental hydrogel composition of claim 8, wherein the moieties are crosslinked.

14. The bilayer dental hydrogel composition of claim 8, wherein the composition exhibits at least one of:
shear thinning;
an in vivo degradation profile of more than 4 weeks and less than 6 months;
a degree of dopamine conjugation that is between 5 wt. % and 35 wt. %.

15. A method of encapsulating a gingival mesenchymal stem cells in a dental hydrogel composition comprising:
(i) disposing the gingival mesenchymal stem cells in the composition of claim 1; and
(ii) exposing (i) to light so that the moieties are crosslinked.

16. A method of delivering gingival mesenchymal stem cells to periodontal tissue, the method comprising forming a bi-layered composition by:
forming a first layer of the bi-layered composition by:
(a) disposing TGF-B3 in the composition of claim 1;
(b) contacting the composition of (a) with periodontal tissue;
(c) crosslinking the moieties that are crosslinkable upon exposure to light by exposing the composition of (b) to light.

17. The method of claim 16, further comprising:
forming a second layer of the bi-layered composition by:
(d) obtaining gingival mesenchymal stem cells;
(e) disposing the gingival mesenchymal stem cells in a hydrogel composition comprising, the hydrogel composition:
(i) polysaccharide;
(ii) polydopamine conjugated to the polysaccharide, wherein between 5 and 35% of polysaccharide sugar moieties are conjugated to polydopamine;
(iii) RGD peptide coupled to the polysaccharide-polydopamine conjugate; and
(iv) moieties that are crosslinkable upon exposure to light coupled to the polysaccharide;
wherein components (i)-(iv) are disposed in the hydrogel composition such that the hydrogel composition:
exhibits an adhesive strength of at least 10 kPa upon cross linking of crosslinkable moieties; and
exhibits an elasticity between 5 kPa and 100 kPa upon cross linking of crosslinkable moieties;
(f) contacting the hydrogel composition of (e) with the first layer; and
(g) crosslinking the moieties that are crosslinkable upon exposure to light by exposing the composition of (f) to light;
thereby delivering the gingival mesenchymal stem cells to the periodontal tissue.

18. The method of claim 17, wherein delivering the gingival mesenchymal stem cells to periodontal tissue regenerates the periodontal tissue.

19. The method of claim 17, wherein the gingival mesenchymal stem cells exhibit more than 60% in vitro differentiation when disposed in the composition.

20. A method for regenerating periodontal ligament-like and osteogenic tissues in a subject in need thereof, the method comprising:
(a) contacting a first layer of the bilayer dental hydrogel composition of claim 8 with periodontal tissue of the subject,
(b) exposing the first layer of the bi-layered hydrogel composition to light to crosslink the crosslinkable moieties;
(c) contacting the light exposed first layer of (b) with the second layer of the bilayer dental hydrogel composition; and
(d) exposing the second layer of the bi-layered hydrogel composition to light to crosslink the crosslinkable moieties.
